(19) United States
(12) Patent Application Publication
(10) Pub. No.: US 2017/0173059 A1
(43) Pub. Date: Jun. 22, 2017

(54) Novel Combination of Naturally Occurring Compounds to Assist With Suspected Mycobacterial Infections Related to Autoimmune Conditions

(71) Applicant: Nicholas Magrone, JR., Orlando, FL (US)

(72) Inventor: Nicholas Magrone, JR., Orlando, FL (US)

(21) Appl. No.: 15/354,975

(22) Filed: Nov. 17, 2016

Related U.S. Application Data
(60) Provisional application No. 62/386,996, filed on Dec. 17, 2015.

(51) Int. Cl. A61K 31/704; A61K 31/216; A61K 31/085; A61K 31/045; A61K 36/54; A61K 31/05; A61K 31/015; A61K 36/53; A61K 31/105; A61K 31/198; A61K 36/8962; A61K 31/56; A61K 36/23; A61K 31/4375; A61K 31/473; A61K 36/29; A61K 36/324; A61K 31/07; A61K 31/593; A61K 9/48; A61K 9/00; A61K 9/20; A61K 31/11

(52) U.S. Cl. CPC A61K 31/704; A61K 31/11; A61K 31/216; A61K 31/085; A61K 31/045; A61K 36/54; A61K 31/05; A61K 31/015; A61K 36/53; A61K 31/105; A61K 31/198; A61K 36/8962; A61K 31/56; A61K 36/23; A61K 31/4375; A61K 31/473; A61K 36/29; A61K 36/324; A61K 31/07; A61K 31/593; A61K 9/4825; A61K 9/4891; A61K 9/4816; A61K 9/0095; A61K 9/20; A61K 2236/331; A61K 2236/33

(57) ABSTRACT

The novel aspect of this invention is that it is a unique formulation of naturally occurring compounds with a unique intention to assist the concerns associated with autoimmune conditions, mainly inflammatory bowel disease (collectively Crohn's, ulcerative colitis, irritable bowel syndrome), Type 1 diabetes, Multiple sclerosis, Psoriasis and the suspected infection of the pathogen Mycobacterium avium sub-species paratuberculosis (MAP). Furthermore, its intent is to address the lack of treatment options available for those with aforementioned autoimmune conditions. Specifically, to offer an alternative option that does not pose the amount of negative side effects that may be associated with contemporary treatment methods. The invention will offer a treatment method that is lower in cost as persons with autoimmune conditions conventional treatment methods costs are rising even under most insurance provider's coverage. In addition, the invention offers an available method without the barriers to care most people with autoimmune conditions may encounter. The invention will provide educational information to persons with autoimmune conditions to offer an individual choice in methods of treatment. The invention will offer persons with autoimmune conditions a variety of options as said invention can be utilized con-currently with conventional treatment methods.

### BACKGROUND

The Field of the Invention

This invention relates to naturally occurring compounds from botanically based chemicals with two fat soluble nutrients to address potential infection from Mycobacterium avium sub-species paratuberculosis. (MAP) Further, the compounds and methods address chronic inflammation associated with autoimmune conditions that are associated with infection from Mycobacterium avium sub-species paratuberculosis. The invention relates to the field(s) of pharmacology, pharmacognosy, microbiology, alternative medicine, allopathic medicine, veterinary medicine or any field of similar discipline.

Background Art

This novel compound is designed to assist with Mycobacterial infections related to autoimmune conditions, specifically inflammatory bowel disease (IBD) which includes Crohn's disease, colitis and irritable bowel syndrome. Recent research has suspected an intracellular agent, Mycobacterium avium subspecies paratuberculosis (MAP), to play a role in the development of autoimmune conditions. (Chiodini, R., Chamberlin, W., Sarosiek, J., & McCallum, R. (2012). Crohn's disease and the mycobacterioses: A quarter century later. Causation or simple association? Crit Rev Microbiol, 38(1), 52-93. doi:10.3109/1040841X.2011.638273.) Contemporary treatment methods for autoimmune conditions, specifically IBD, consist of corticosteroids, immune modulating agents, 5-ASA compounds and other anti-inflammatory agents. These treatment methods do not address the suspected MAP infections that may be related to IBD.

This invention is a formulation of naturally occurring compounds that offers an alternative to contemporary treatment of suspected MAP infections. There have been recent developments in contemporary medicine utilizing multiple synthetic anti-microbial agents that are designed to target MAP. (Please refer to http://www.bioworld.com/content/redhill-start-phase-iii-antibiotic-therapy-crohns-0 and http://www.redhillbio.com/rhb-104). These synthetics may have detrimental effects to patients as suggested length of treatment ranges from 9 months to several years. Possible detrimental effects may include damage to the liver and cardiovascular system, alteration of micro-flora and other negative effects to metabolic processes. This formulation of naturally occurring compounds provides a potential alternative method to address suspected MAP infections without the detrimental side effects of contemporary methods.

### BRIEF SUMMARY OF THE INVENTION

The formulation of this invention consists of Cinnamon oil active compounds and derivatives (Cinnamomum verum), active compounds from Wild Mountain Oregano (Origanum vulgare), compounds active in Gotu Kola (Centella asiatica) species, Vitamin A and its metabolites, the hormone classified as 25-hydroxy-D3 otherwise known as Vitamin D3, Garlic (Allium sativum) and its active compounds, Oregon Grape (Mahonia aquifolium) and its active compounds, and Boswellia serrata resins and its active compounds. The formulation is selected due to these natural agents' qualities that collectively consist of anti-microbial properties, anti-inflammatory properties, tissue regenerative properties and immune system enhancing properties. This summary provides the formulation's beneficial properties as a whole and the detailed description section provides an in-depth description of the individual compounds listed.

### BRIEF DESCRIPTION OF THE DRAWINGS

Not Applicable

### DETAILED DESCRIPTION OF THE INVENTION AND EMBODIMENTS

### Cinnamon Oil and Derivatives 300 mg

Cinnamon (cinnamomum verum, commonly known as Ceylon) is a globally recognized spice that has been used for decades in the preparation of foods. Its writings and references can be traced as far back to Biblical time eras as described in the book of Proverbs and parts of the Hebrew Talmud. The family of plants it is classified under is Lauraceae which are more commonly referred to as Laurels. Mainly native to tropical regions, Cinnamon is readily found in Southeast Asia and South America in high concentrations. Its translation in native countries can be described as “sweet bark” or “sweet wood” as well as “tube” or “cylinder” in some Latin areas. Cassia usually designates the part of the plant that is used most which is the bark or outer peelings of a species of tree—Cinnamomum iners. Documentation of the Cinnamon plant has been found in descriptive manners in ancient Rome, Greece, and Egypt in preparations of local foods and events.

As mentioned above, the most widely recognized family of Cinnamon is Cinnamomum verum or Ceylon. Cinnamon has many different lineages and other common species used throughout the world are C. burmannii, C. loureiroi, and C. cassia. The differences in each family are reflected in the texture of bark, the layers of bark utilized, and the flavor. C. cassia is most likely reflected as the strongest “flavor” while Ceylon has a denser aromatic availability. This could partly be due to utilization of different areas or layers of bark and changes in respective volatile oils and polyphenolic compounds.

The active compounds found in most species of Cinnamon differ. But its strong scent and taste can be directly related to several polyphenols and aldehydes. Cinnamaldehyde and Trans-Cinnamaldehyde are the main compounds that are found in the oils of the bark of the plant when pressed into the essential oil form (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990). Other compounds found in the volatile oil from Cinnamon species are Ethyl cinnamate, Eugenol, Methyl chavicol and Linalool aldehyde. The actions of the aldehydes and compounds are discussed further in this document.

Because of its many active constituents, Cinnamon has been attractive for a wide application of uses beyond the food industry. It has been used in pesticide applications for a limited amount of areas that express interest in applying an agent that does not have the drawbacks of residues that can become toxic over time. In other small areas of interest, its compounds have been used for the issue of disease vectors such as several species of mosquito larvae. Another use of Cinnamon has been to preserve food items that would normally be readily disposed of due to spoilage. The oils and compounds active in the plant have a specific action in the level of glucose that is problematic for adult onset diabetes mellitus. The mechanism is most likely due to its activity on insulin receptor sites and its related effects to protect receptors from damage or oxidation from free radicals. Research has also proven its effects on the more important measurement of Hemoglobin A1c in type 2 diabetes. This measurement may be a more accurate method of testing for its relation to glucose tolerance and insulin sensitivity. Other research suggests Cinnamon has a wide potential of use in action against select viruses. The antioxidant capacity of its compounds has been recently recorded for the potential use in action against animal melanoma in vitro samples.

For the application of this text, Cinnamon has a large potential for its antibacterial possibilities. The aldehydes and oil extracts from the family of Cinnamon have been utilized for their action against many different pathogens (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990.). Cinnamon, along with a wide variety of other well-known spices, has shown action specifically against problematic strains of bacteria. Recorded strains the compounds and aldehydes have been shown to inhibit are Campylobacter jejuni, E. Coli O157-H7, Listeria monocytogenes and Salmonella enterica (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990). Along with other naturally occurring compounds found in spices, Cinnamon oil extract has been demonstrated to inhibit the nosocomial pathogens in the family of Staphylococcus aureus (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990). The compounds have also been found to be active against strains of problematic fungal agents such as Candida albicans. This is important as a base example to apply the compounds tested against the pathogens mentioned for contemporary research that is not currently being investigated to its potential.

The pathogen that pertains to this text is Mycobacterium avium subspecies paratuberculosis. MAP (abbrv.) is in the family of Actinobacteria and is closely related to well-known “household” related Mycobacterium such as M. tuberculosis, M. leprae and M. avium. The actions and behaviors of these pathogens are vastly different from non-spore forming bacteria. This has been displayed in public health measures to protect the public against mycobacteria. Many disparities can be associated with many different strains of Mycobacteria. As history has shown, the effects of this class of bacteria are devastating and are resulting in harmful effects to public health with consequences in all areas of civilization. The efforts have been directed from a scientific, medical, and ethical standpoint and resulting success recorded are not surprising when an understanding of how this class of pathogens acts in hosts and in the respective environment.

Mycobacteria are slow to divide, troublesome to culture, and extremely difficult to remove from affected hosts. Human exposure to mycobacteria is usually communicable and has similar features from each strain and its related condition. The most widely known characteristic is their ability to invade cells to elude the host's initial immune response. Most mycobacteria, once active in a selected host, can be found in structures called granulomas. These structures are essential to the host's response to house or “wall-off” active mycobacteria that are directly related to tissue damage. The process in which this occurs is most likely a result of the pathogen's ability to evade primary immune response and the ability to culminate in macrophages and possibly lay dormant there. Mycobacteria are also well known for their durable cell wall features that consist of lipid based molecules. This is directly related to the issues of resistance in tuberculosis and what is widely known as XDR-Tb (Extensively Drug Resistant tuberculosis).

The use of selected synthetic antibiotic agents has been directly linked to the topic of resistance. Once the pathogen is exposed to a selected agent, it has the potential to replicate with new DNA that respond to the selected antibiotic agent and build its own defense against it. After reproduction of bacterial cells occurs, the agent is no longer active against those strains of pathogen. This can result in unsuccessful treatment protocols.

The protocols for treatment of mycobacteria are very difficult for most physicians to prescribe and for most patients to adhere to. This is due to long treatment periods that range anywhere from 12 months to 5 years or more, depending on the case. Access to synthetic agents has been a global issue for many decades and it seems it will continue to be an issue for many decades to follow. Monitoring of effectiveness and safety of agents is crucial for positive results and correct clearance of pathogens. This leaves gaps in the areas of research and concerns for consumers who need to select specific agents if exposure has occurred.

The volatile oils and aldehydes of Cinnamon oil have displayed inhibition of MAP in vitro (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990.). Several strains have been tested against its main compounds of Cinnamaldehyde and Trans-cinnamaldehyde and they were shown to inhibit the selected strains of MAP tested in vitro (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990). The methods of action were most likely due to interruption of DNA synthesis in the pathogen, interference of cell enzyme activities, cell membrane disruption, and removal of necessary elements for normal cell function. These methods discussed show multiple positive areas of interest in the preparation of agents for antimicrobial use against difficult pathogens such as MAP. In comparison, synthetic agents are usually limited to one or two functions against pathogens. Whereas compounds found in Cinnamon oil extracts have multi-faceted actions against pathogens due to its many different chemical structures. Synthetic agents (Isoniazid, Rifampin, Rifabutin, Ethambutol, certain fluoroquinolones, and more recently macrolide class antibiotics) that are used against mycobacteria are less safe, especially when the class of agents needs to be used for long durations. What is concerning as well is the misuse of commonly prescribed antibiotics (Amoxicillin, Metronidazole, etc.) that show little to no action against the family of mycobacteria. Cinnamaldehyde and trans-cinnmaladehyde have been listed as GRAS (Generally Regarded as Safe) on material data safety sheets. Compounds found in Cinnamon bark and its extracted oils, do not pose the same safety concerns as synthetic agents, even if usage is for the same duration as synthetics (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990).

Cinnamon oil and its active compounds have displayed action against MAP and other virulent pathogens in vitro. While this novel application for treatment and protection measures is foreign to science, medicine, and agribusiness, its selection for the methods discussed in this product are well suited for future compounds that can be utilized for potential treatment options and eradication efforts. The intent is to be applied in the natural health industry, pharmaceutical industry, and possibly in agriculture/veterinary applications as well.

### Oregano Oil 150 mg Yielding 75-80% Carvacrol

Compounds Occurring in Wild Mountain Oregano (Origanum vulgare)

There are several species of plants that could be classified as what is most commonly known as Oregano. The plant Origanum vulgare or Wild Mountain Oregano is a globally utilized spice for the preparation of food items and is used in many different cultures throughout the world. Oregano is in the family of plants that would be termed “mints” under the taxonomy Lamiaceae. Other names for the herb would be Wild Marjoram. It is native to temperate areas in the Mediterranean region but is also found in warm areas such as the southwest region of Eurasia.

Oregano is a perennial herb that produces violet to light pink colored flowering tops and usually does not thrive well in cooler seasons. Oregano is best suited for arid warm to hot climates as its native lands offer to this versatile herb. The phylogeny like many other herbs has been influenced by humans for different flavorings and utilizations. The most prevalent species used in today's age is Origanum vulgare. Other commonly used species are O. gracile and O. hirtum.

The species variations yield slight differences of phenolic compounds and volatile oils that provide different flavors and aromas. The active compounds in the family of Oregano that are used for many applications are carvacrol, thymol, caryophyllene, limonene and rosmarinic acids. The advanced metabolites and constituents or “terpinoids” that are active in the plant are p-cymene, spathulenol, beta-fenchyl ether alcohols, and germacrene-D. For the purposes of this text, these compounds are the naturally occurring elements that will be discussed regarding how they can be applied to the said product.

While this species of plant and its compounds have been found to be useful in many different applications, the food industry is the most common. However, Oregano and its compounds have also been used in applications for pesticide treatment and its benefits are obvious in that application. Most synthesized pesticide compounds have residuals that may have toxic metabolites and toxic active compounds. This gives herbal based compounds like Oregano an advantage as it is classified as GRAS in its wide variety of uses. The volatile oils have also been utilized for food preservatives and have been used in the process of curing meats.

The intent and purpose of said product is to utilize these active compounds (mainly carvacrol) for its potential antibacterial mechanisms, as well as its antioxidant capacity. Oregano has been used for many decades as a potent antibacterial agent. Some documentation dates to a time when Hippocrates selected this species for use against contagious pathogens. Oregano has also been used as purative agent or “bitters” for the digestive tract throughout history as well. The largely studied compound that is found in high concentrations of Oregano is carvacrol. This flavonoid or compound is best active when its percentage found in the strain of plant is between 70-80%. Carvacrol has displayed it effects on certain pathogens in a wide range of scientific research (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990.). It also has a wide range of action that may disrupt cell replication and display potential mechanisms that disrupt outer membrane of pathogens (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990.). This is highly interesting as most synthetic antimicrobial agents have been rendered non-effective as evolving strains of bacteria are now resistant to widely over utilized agents.

This is more important if the pathogen is in the mycobacterial family. As stated, the purpose of this text is to select active compounds in naturally occurring elements and its potential use for the pathogen Mycobacterium avium subspecies paratuberculosis (MAP abbrv.).

MAP has been highly suspected in developed nations for its potential to become a zoonotic agent. MAP is the primary agent responsible for the ruminant disease termed Johne's disease. Johne's is a chronic, granulomatous creating condition that occurs mainly in cattle but occurs in the entire family of mammals in the genus ruminate or ruminants. Manifestations are marked elevation of fecal shedding, weight loss, drop in production of milk, and eventually herd losses (Collins, M., Cho, D., & Sung, N. (2006). Identification of proteins of potential diagnostic value for bovine paratuberculosis. Proteomics, 6(21), 5785-5794.). The nations in the EU have led the way in research surrounding MAP, mainly due to its economic impact of the dairy industries in select countries. MAP does not have required standards for safety currently in the United States as it pertains to public health.

MAP can have a dormancy period in mammalian infections. This could explain the science that shows newborn calves do not test positive for PCR testing or ELISA samples. Cattle may spread the pathogen throughout feed lots by shedding bacterial loads onto soil, feed supplies, and newborn calves. If it is not obvious enough that the potential of this pathogen is an intracellular agent, the case should be made that MAP may be involved in placental transmission. MAP is able to survive current retail measures of pasteurization and has been readily cultured from retail dairy products and retail beef (Collins, M., Wells, S., Petrini, K., Schultz, R., & Whitlock, R. (2005). Evaluation of Five Antibody Detection Tests for Diagnosis of Bovine Paratuberculosis. CVI, 12(6), 685-692.). This brings up the comparison of Johne's disease and the collective group of inflammatory bowel disease conditions named Crohn's disease and ulcerative colitis. In comparison, both ruminant disease and human manifestations share the same symptoms. MAP has also been suggested as a focal point of research for other autoimmune diseases.

This brings up the necessary research for an introduction of contemporary antimicrobial agents that offer a greater yield of benefits without the issue of access, potential side effects, and greater difficulty with compliance. Herbs like Oregano offer potential compounds that have displayed action against MAP in vitro and a wide variety of other bacteria that may be co-infectious agents (Wong, S., Grant, I., Friedman, M., Elliott, C., & Situ, C. (2008). Antibacterial Activities of Naturally Occurring Compounds against Mycobacterium avium subsp. paratuberculosis. Applied and Environmental Microbiology, 5986-5990.).

Oregano and its compounds carvacrol and thymol appear to possess actions similar to classical agents used in tuberculosis therapy such as Isoniazid and Rifampin. Both share the ability to penetrate the waxy, lipo-dense outer layer of mycobacteria. MAP shares this behavior with other mycobacteria. This makes treatment and therapies difficult as often with other mycobacteria they are slow to replicate and highly capable of producing resistance to synthetic agents. Carvacrol and the wide range of flavonoids are powerful compounds that possess the potential to address issues that MAP poses to public health.

In addition to the wide antibacterial applications, Oregano has a very high ORAC (oxygen radical absorbance capacity) rating. In terms of ORAC ranges, Oregano species antioxidant ratings have been scaled at 175,295 μmol/TE. (Nutrient Data: Oxygen Radical Absorbance Capacity (ORAC) of Selected Foods, Release 2 (2010). Value is displayed in moles of Trolox Equivalents per 100 gram sample. In comparison to select “superfoods” this value is shown to be close to a two to three-fold or even higher yield than other antioxidant dense herb and food items. Research has utilized the capacity of the species of Oregano in industrial applications for reducing use of synthetic antioxidant compounds for storage of perishable items.

The further benefits of the compounds that pertain to this text are the potential anti-inflammatory mechanisms Oregano compounds display. In reflected research, critical extracts of Oregano oils displayed a dose-dependent reduction of pro-inflammatory cytokines TNF-α, IL-1β, IL-6 (Ocaña-Fuentes, A., Arranz-Gutiérrez, E., Señorans, F., & Reglero, G. (n.d.). Supercritical fluid extraction of oregano (Origanum vulgare) essentials oils: Anti-inflammatory properties based on cytokine response on THP-1 macrophages. Food and Chemical Toxicology, 1568-1575.).

This research also displayed an increased yield in the anti-inflammatory cytokine IL-10. While contemporary agents (corticosteroids, NSAIDS, monoclonal based biologics, and purine analogues) that are used in allopathic settings for their inflammatory ameliorating effects are pronounced, potential side effects are to be considered for long term use. This example of research for said compounds is another piece of valuable evidence that pertains to this text in the application potential that targets autoimmune conditions and their possible relation to MAP discussed herein. The intended area of application for said product would be the natural health industry, pharmaceutical industry, and possibly veterinary/agriculture applications (Dorman H J D and Deans S G. Antimicrobial agents from plants: antibacterial activity of plant volatile oils. J of Applied Microbiology. 2000; 88: 308-316.).

### Gotu Kola (Centella Asiatica) 200 mg Yielding Active Asiaticosides

Most commonly known for its ayurvedic applications, Gotu kola or Centella asiatica has a wide variety of traditional uses. It is known under a long list of names under Ayurveda and Eastern medicine such as Indian Pennywort, Brahmi, Indian waterwort, and marsh penny. Its properties are small clover or fan-shaped leaves, little to no aroma, lightly colored flowers, and it produces small round shaped fruit. Gotu kola is most frequently found near dense watery areas.

Gotu kola is an aquatic perennial creeping plant closely related to the family of parsley species. It is most commonly found in regions of India, South Africa, Madagascar, Sri Lanka, Australia, Indonesia, and areas of China.

The uses for Gotu kola are very diverse throughout history. It has been prepared as a traditional side dish in Indian culture and is usually served with different types of rice and legumes such as Dal. It is also commonly prepared with various curry and chili powders. Many other nations, such as Sri Lanka, prepare Gotu kola in the same manner. Other countries in the same regions serve Gotu kola as a beverage similar to the way in which various teas are prepared.

Gotu kola may have an even broader application of use in the realm of medicine. Traditionally, it has been used for dermal lesions and conditions of the skin. It has been well documented throughout history in its treatment of advanced conditions that affect the skin such as leprosy and psoriasis (Brinkhaus, B., Lindner, M., Schuppan, D., & Hahn, E. (n.d.). Chemical, pharmacological and clinical profile of the East Asian medical plant Centella aslatica. Phytomedicine, 427-448.).

Its anxiolytic properties have also been vastly researched as a potential agent that would be of benefit. Many of the compounds discussed herein may be responsible for positive effects in treatment of anxiety related disorders (Sarris, J., Mcintyre, E., & Camfield, D. (n.d.). Plant-Based Medicines for Anxiety Disorders, Part 2: A Review of Clinical Studies with Supporting Preclinical Evidence. CNS Drugs, 301-319.). It should be noted that Brahmi in Ayurveda is labeled to bring forth knowledge and is widely used to regenerate nerve function. Centella species have also been used in the treatment of circulatory conditions and is widely used for cosmetic therapies such as treatment of varicose veins. Along with cosmetic uses, Centella has also been used for the treatment of wound healing and treatment of scar tissue (Pointel, J., Boccalon, H., Cloarec, M., Ledevehat, C., & Joubert, M. (1987). Titrated Extract of Centella Asiatica (TECA) in the Treatment of Venous Insufficiency of the Lower Limbs. Angiology, 46-50.). The mechanisms may partly be due to the Centella species' ability to allow compounds with high antioxidant capacities to be vetted to areas in need of collagen stimulation. The primary compound involved in this mechanism is Triterpinic acid. The antioxidant enhancing effects are directly related to free radical enzyme production of super oxide dismutase and catalase.

Centella species have also been recently researched in regards to cardiovascular intervention. Gotu kola is suggested to help endothelial function as stated above, as well as stimulating production of collagen (Cesarone M R, Belcaro G, Nicolaides A N, Geroulakos G, Bucci M, Dugall M, De Sanctis M T, Incandela L, Griffin M, Sabetai M. Increase in echogenicity of echolucent carotid plaques after treatment with total triterpenic fraction of Centella asiatica: a prospective, placebo-controlled, randomized trial. Angiology 2001 Oct.; 52 Suppl 2:S19-2).

For this text, the antibacterial components will be further discussed. Centella species have been widely used in ancient medicine for a variety of infections. The most common would be the class of mycobacteria responsible for Hansen's disease (also known as leprosy)—Mycobacterium leprae. The pathogen responsible for leprosy behaves very similar to other species of mycobacterium. It is a very hardy, pleomorphic, gram positive, acid fast, aerobic bacilli. Like many other mycobacteria it also has a waxy outer membrane. M. leprae is cultured in longer durations like other mycobacteria (McMurray, D. N. (2000). Mycobacteria and nocardia. Medical Microbiology.).

The use of Centella for Hansen's is most likely due to the wide array of compounds that have direct action against this feature of mycobacteria. The compounds that possibly exhibit actions against pathogens like M. leprae and M. paratuberculosis are but are not limited to, a variety of triterpenes including asiaticoside, madecassoside, Asiatic acids, madessic acids, terminolic acid and asiaticoside-B (Schaneburg, B., Mikell, J., Bedir, E., Khan, I., & Nachname, A. (2003). An improved HPLC method for quantitative determination of six triterpenes in Centella asiatica extracts and commercial products. Die Pharmazie, 58(6), 381-384). These compounds may also give rise to the versatility of Centella species and their potential applications.

Being that Centella species have demonstrated applications for complex conditions such as Hansen's, the potential Gotu kola and related compounds have in pharmaceutical applications for the possible infections and conditions associated with M. paratuberculosis are directly applicable to this formulation. MAP initiates inflammatory mediators and pro-inflammatory cytokines similar to that of M. leprae, therefore Centella plants and their active compounds have a potential utility for creating an agent for therapeutic use. Gotu kola has been associated with healing of dense fibrotic keloid tissue which results in chronic inflammation with a condition such as Hansen's. Since many recent research publications attempt to connect the association of M. paratuberculosis with autoimmune conditions that have similar distinctive features and results like Hansen's (i.e. inflammatory bowel disease, Type 1 diabetes, Multiple Sclerosis and others), Centella species have the potential to address the issues related to chronic inflammation by methods of targeting selected pathogens such as M. paratuberculosis. Previous comparisons of M. leprae and MAP have been established and are noted as data lends to the theory of both pathogens sharing similar features (Greenstein, R. (2003). Is Crohn's disease caused by a mycobacterium? Comparisons with leprosy, tuberculosis, and Johne's disease. The Lancet, 3(8), 507-514.). It should be emphasized that Gotu kola promotes healing and repair of damaged tissue that may have resulted in chronic, long term injury. Research findings related to Gotu kola as outlined above, provide evidence that it may be directly used in the novel treatments of Crohn's disease and ulcerative colitis. Since the two collectively termed inflammatory bowel diseases may often result in fibrotic dense tissue occurring in isolated regions of the digestive tract, Gotu kola is an agent that may promote healing and repair of damaged tissue.

One other important reason Gotu kola may have potential to be utilized in conditions hypothesized to be initiated by MAP, is due to the low selection of synthetic antibacterial agents that have displayed inhibition. A reason for this may be the lack of ability to penetrate the dense outer membrane of mycobacterial species. As mentioned previously, Centella asiatica has displayed mechanisms that have strong potential influence on the lipid dense membrane of the mycobacterial family, MAP being included. With unaffordable synthetic compounds and barriers in the medical industry to correct underlying issues such as potential infectious agents perpetuating autoimmune conditions, alternative options are necessary.

### Vitamin a and Metabolites (as Beta Carotene) 4000 IU

The term Vitamin A refers to a group of fat soluble chemical structures which are collectively read as:
(2E,4E,6E,8E)-3,7-Dimethyl-9-(2,6,6-trimethyl-cyclohex-1-enyl)-nona-2,4,6,8-tetraen-1-ol
(Retinol)

The primary compounds that classify as vitamin A are retinoic acid, retinol, retinal, and a group of provitamin compounds. When found in animal food sources, vitamin A is usually defined as retinyl palmitate. Once conversion is completed, the retinol form of the vitamin is usually stored for later use or conversion to an aldehyde.

The groups collectively have a chain which may be classified as a “retinyl group”. These structures are necessary for the functions of the vitamin. The popular carotenoid beta carotene is widely recognized for its color components it provides to fruits and vegetables, most notably to carrots.

The forms found in foods are broken into two categories. The first form is the animal source which is eventually converted into retinol. This is a fat-soluble compound that is marketed in commercial products and nutrition labels as retinyl palmitate. The second form is the carotenoid form that is found in foods, mainly the carotenes beta, alpha, and gamma. Another compound of vitamin A is the group of xanothophyll structures.

The most widely recognized utilization of this group of compounds in the realm of nutrition is in a wide variety of optical functions, including providing color to vision. Vitamin A is also widely noted for its functions in helping immune function. It has been recognized as giving growth factors to epithelial cells as well (Tanumihardjo, S. (2011). Vitamin A: Biomarkers of nutrition for development. American Journal of Clinical Nutrition, 658S-665S).

There are a large variety of foods that vitamin A and its compounds can be found in, a lot of them yielding high amounts. Some of those are cod liver and many other animal liver preparations, dandelion, tomato, broccoli, kale and many other greens. As mentioned before, vitamin A and its compounds are vital in many necessary functions and processes in biology such as immune function, genetic transcript processes, antioxidant processes, integumentary function, bone matrix processes, embryo development function and vision (Combs, Gerald F. (2008). The Vitamins: Fundamental Aspects in Nutrition and Health (3rd ed.). Burlington: Elsevier Academic Press. ISBN 978-0-12-183493-7. Duester G (2008). “Retinoic Acid Synthesis and Signaling during Early Organogenesis”. Cell 134 (6): 921-31. doi:10.1016/j.cell.2008.09.002.PMC 2632951. PMID 18805086. Nelson A M, Zhao W, Gilliland K L, Zaenglein A L, Liu W, Thiboutot D M (2008). “Neutrophil gelatinase-associated lipocalin mediates 13-cis retinoic acid-induced apoptosis of human sebaceous gland cells”. The Journal of Clinical Investigation 118 (4): 1468-1478. doi:10.1172/JCI33869. PMC 2262030.PMID 18317594.).

For the application intended herein, vitamin A is a vital component that influences immune function and has a high antioxidant effect on cellular functions. In addition, the recent review of vitamin A and metabolites have been found to inhibit strains of mycobacterium in vitro including Mycobacterium ss. Paratuberculosis (MAP) (Greenstein, R., Su, L., & Brown, S. (2012). Vitamins A & D Inhibit the Growth of Mycobacteria in Radiometric Culture. PLoS ONE. Retrieved from www.plosone.org). Since the hypothesis that was proposed many decades ago that suggests that MAP is directly related to autoimmune conditions, there has been little effort to address this issue by means of synthetic or naturally occurring compounds.

Vitamin A is a prime example that can be directly applied to address an issue as expansive as autoimmune conditions. The manners in which autoimmune conditions are addressed currently do not improve immune function and regrettably are most often treated with immune suppressing agents like corticosteroids and purine inhibitors among many other compounds. If addressing a condition that was possibly initiated by a strong influencer of metabolic functions and inflammatory segues such as MAP are treated by suppressing the immune system, the outcomes will largely be negative. Being that vitamin A has a strong influence on immune function, antioxidant functions, and from the example shown above, it has a potential to be directly applied to mycobacterial agents (Greenstein, R., Su, L., & Brown, S. (2012). Vitamins A & D Inhibit the Growth of Mycobacteria in Radiometric Culture. PLoS ONE. Retrieved from www.plosone.org). This proves that its intention for this application relates quite well.

Vitamin A and compounds show in recent experimentation, the effect of in vitro inhibition to several strains of MAP and several other closely related mycobacterial family members (Greenstein, R., Su, L., & Brown, S. (2012). Vitamins A & D Inhibit the Growth of Mycobacteria in Radiometric Culture. PLoS ONE. Retrieved from www.plosone.org). The precursors and metabolites shown in experiments were β carotene, retinyl acetate, retinoic acid, and 13 cis-retinoic acid.

The strains of MAP that were tested against these compounds were that of bovine origin and human strains obtained from serum samples (Greenstein, R., Su, L., & Brown, S. (2012). Vitamins A & D Inhibit the Growth of Mycobacteria in Radiometric Culture. PLoS ONE. Retrieved from www.plosone.org). All strains displayed dose dependent inhibition in sample testing using control agents (Monensin) that were applied and have displayed inhibition of MAP in the prevention and treatment of Johne's disease. During testing, Retinol showed a strong inhibitory effect of all strains of MAP, M. avium, and M.Tb (Greenstein, R., Su, L., & Brown, S. (2012). Vitamins A & D Inhibit the Growth of Mycobacteria in Radiometric Culture. PLoS ONE. Retrieved from www.plosone.org). The other selected compounds and metabolites of vitamin A (Retinoic acid, retinyl acetate, β carotene, and 13 cis-retinoic acid) showed inhibitory effects with retinyl acetate showing the highest inhibition of the group (Greenstein, R., Su, L., & Brown, S. (2012). Vitamins A & D Inhibit the Growth of Mycobacteria in Radiometric Culture. PLoS ONE. Retrieved from www.plosone.org).

In terms of application pertaining to this text, vitamin A and its metabolic compounds and derivatives have shown a potential for use in autoimmune therapy. This is widely applicable for many areas of industry in science and medicine as current methods do not favor long term use of agents like corticosteroid compounds, monoclonal antibody based biologics, and purine inhibitory class drugs. With the strong effect of down regulation of immune function and output, the agents mentioned do not favor a patient who has a low immune response to a potential infection like MAP. The addition of a strong free radical scavenger, immune enhancing, and possible anti-mycobacterial agent like vitamin A is important to consider as an option for addressing autoimmune conditions.

Due to current issues with food standards and genetic alteration of food-based plants, the effect on necessary compounds like vitamin A are important to research. As dietary compliance is a difficult topic to address with many areas of science and medicine, the addition of a class of necessary nutrients like vitamin A through supplementation should be noted. The food industry is showing a decline in vital nutrients and vitamin A proves to be a “natural” agent that is of importance and will also prove to be important in the application that this text outlines.

### Vitamin D3 (as Cholecalciferol) 1500 IU

Vitamin D3 is commonly termed a necessary “vitamin” that is required for absorption of vital minerals and nutrients, most notably calcium, iron, magnesium, phosphorus and many other widely used compounds in the body. Vitamin D3 is closer to being a hormone compound rather than a vitamin as the body can synthesize D3 via hepatic processes after skin exposure to direct sunlight. It is usually classified as two compounds that can be collectively termed vitamin D—cholecalciferol as D3 and ergocalciferol as D2. Both compounds can be obtained via food items or supplemental use.

Vitamin D3 is grouped in a class of fat soluble steroid based chemicals. The fact that humans can synthesize D3 as 25-hydroxyvitamin D3 upon skin exposure to sunlight make it a semi-essential nutrient. Being that D3 is involved in many important metabolic functions, it is a very important compound. D3 is classically recognized for its role in prevention of Rickets. Many other conditions are controlled by adequate levels of D3. The issue that seems to arise is controlling the serum concentration of D3 at recommended levels. This can be an issue in geographic terms as areas that have lower levels of sunlight can lead to lower levels of D3 in serum samples.

Bone health is the most widely marketed area of health that D3 is recognized in. In terms of this text, the other effects that D3 has on health and wellness will be discussed. Low levels of D3 are repeatedly correlated with chronic conditions. There are many factors to consider but in the aspect of how an illness is initiated, it is easy to understand how low levels of 25-hydroxy D3 may play a role (Feelisch, M., Gorman, S., & Weller, R. (n.d.). Vitamin D status and ill health. The Lancet Diabetes & Endocrinology, E8-E8.).

There is a fine line in determining how much D3 should be consumed to obtain its optimal effects. Excess levels of D3 in short term use does not appear to influence biological functions (Tuohimaa, P. (n.d.). Vitamin D, aging, and cancer. Nutrition Reviews, S147-S152.).

Long term use of higher concentrations of D3 may have drawbacks, but it is difficult to obtain a constant level of biologically available 25-hydroxy D3. Many environmental processes utilize D3 rapidly. Lack of adequate sleep, chronic stress, and chronic health conditions all contribute to D3 depletion. In these states, use of D3 for short term in higher amounts may assist with biological functions. D3 has been utilized in some areas with success in treatment of various types of cancer (Byers, T. (2010). Anticancer Vitamins du Jour—The ABCED's So Far. American Journal of Epidemiology, 1-3.). Other conditions that D3 has been suggested to influence are mental health, cardiovascular conditions, fertility, and most important to this text, autoimmune conditions.

In autoimmune conditions, many vital nutrients are depleted. The process that creates this environment is a complex one. Many times, the argument is made that low levels of compounds like vitamin D3 and other important nutrients create conditions. Others suggest that the condition itself is responsible for low levels of vital nutrients. Whichever side the argument stems from, it is always important to note how the compounds and nutrients improve or favor a positive response to the condition.

Autoimmune conditions are one area that constantly reflects low markers of important compounds and nutrients. Vitamin D3 is an important factor that is involved in these conditions. The cause of autoimmune conditions is still under debate. Recent hypothesis has suggested that Mycobacterial infections are related. The specific pathogen that is suggested is Mycobacteria subspecies paratuberculosis (MAP). As discussed previously, MAP has a strong influence on inflammatory markers that are directly related to autoimmune conditions. The focal point of research surrounding MAP has been around inflammatory bowel conditions. The two specific conditions are ulcerative colitis and Crohn's disease.

Research that has used samples of MAP in vitro against fat soluble vitamins, specifically vitamin D3, has shown inhibition of isolated strains from bovine sources and human isolates (Greenstein, R., Su, L., & Brown, S. (2012). Vitamins A & D Inhibit the Growth of Mycobacteria in Radiometric Culture. PLoS ONE. Retrieved from www.plosone.org).

The examples of inhibitory effects of fat soluble nutrients like vitamin D3 are applicable for this text in a similar manner as vitamin A and its metabolites. The examples of inhibition are an added bonus in addition to the many other effects of D3 which show influence of positive response to chronic disease. Vitamin D3 has a direct effect on the immune system and has demonstrated specific anti-neoplastic activity in colon tissue. Patients with inflammatory bowel disease (IBD) are at a higher risk for colon, esophageal, and other various types of cancer. The argument around whether those with IBD have an over-active immune response or a lower immune response to a particular pathogen, like MAP, will be under debate for decades. When a compound like D3 can address all areas of question in autoimmune conditions that may be related to an infectious agent, the benefits far outweigh the questions surrounding nutraceutical agents and their positive influence on chronic conditions. D3 may have a synergistic effect in combination with other fat soluble nutrients like vitamin A and its metabolites (Greenstein, R., Su, L., & Brown, S. (2012). Vitamins A & D Inhibit the Growth of Mycobacteria in Radiometric Culture. PLoS ONE. Retrieved from www.plosone.org).

As it relates to the product mentioned herein, D3 should be noted as an important compound for those who may have lower levels of 25-hydroxy D3 as its functions have a positive effect on the immune system and may have an inhibitory action against suggested infectious agents such as MAP. As an added benefit, D3 has been shown to protect against neoplastic development in colon tissue. This is an important function as persons with a condition such as ulcerative colitis or Crohn's disease may be at a higher risk for related tumor growth. It is also important to understand that people who are currently treated for these conditions may have been prescribed corticosteroids and agents that suppress the immune system like 6-Mercaptopurine and Azathioprine. These collective agents significantly decrease immune function. The side effects of corticosteroid use can increase risk for joint and bone problems. The long-term use of these agents has been shown to have an increase in fracture rates in clinical settings (Bollet, A., Black, R., & Bunim, J. (1955). Major Undesirable Side-Effects Resulting From Prednisolone And Prednisone. JAMA: The Journal of the American Medical Association, 459-463.).

The pertaining facts surrounding D3 in this text are directly applicable to the product discussed as positive effects from the hormone based compound can assist patients with autoimmune conditions. This is not limited to the primary condition but also an overall increase in wellness from the scientific facts mentioned above. Garlic (Allium sativum) 400 mg yielding active compounds S-Allycysteine, Allicin and Ajoene.

With its long history of use dating back many thousands of years ago, Allium sativum or Garlic has been used for a wide range of applications in scientific history. Culinary use is the most common application of Garlic. The less obvious use is in several medicinal applications. The uses will be discussed herein along with a brief background to its compounds and its potential.

The Ancient Egyptians were known to use Garlic for its benefits long before the compounds that were actively being used were understood. It was labeled “Russian Penicillin” at one point because Russian soldiers applied it in field dressings for infection. It has also been noted in folklore and mythology for many of its benefits by European and Asian nations. Its medical uses have been acknowledged by Hippocrates and many other ancient healers for parasitic infections, respiratory conditions and a long list of other ailments. The name itself was said to be derived from early Anglo-Saxon era definitions of “gar” meaning point or spear-like and “lic” meaning leek, which may also be linked to its family name Lillaceae.

Its genus, Allium, contains a wide array of similar plants like those of onion, leek, and shallot species. It grows around 3-4 feet in height and has a flowering top. While initially grown easier in temperate climates, its pollination from insects has allowed it to be found in almost every nation in one form or another. Garlic reproduces asexually and usually grows from cloves and bulbs of Garlic being planted in soil. Most notably used in European culture for its uses in culinary garnishing, Garlic is found in highest percentages of growth in China. The bulb of the plant is used the most for cooking applications. It is little known that most of the entire plant, including leaves and flowers, are also used for food preparation. It can be easily argued that Garlic is the most widely used herb for culinary application because nations from every continent use it in many of their notably famous dishes.

Garlic has a wide range of nutrients that may explain the reason it can be used for a wide array of conditions. It is found to have high amounts of Vitamin C, B6, B1, B5 and many minerals like zinc, selenium and manganese. Garlic has been used in folk medicine for the early treatment of respiratory illnesses like the common cold, flu, and tuberculosis.

Garlic has been proven in many studies to have a positive influence on a wide range of cardiovascular conditions. Trials have even demonstrated that the Garlic extract has displayed better effects on blood pressure than synthetic agents (Ried, K.; Frank, O. R.; Stocks, N. P. (2010). “Aged garlic extract lowers blood pressure in patients with treated but uncontrolled hypertension: a randomised controlled trial”. Maturitas 67 (2): 144-150.doi:10.1016/j.maturitas.2).

Aged extract of Garlic has been used in research for the treatment of hypertension, hyperlipidemia, and other related conditions. Garlic has a direct effect on platelet aggregation and thus has been compared to several pharmacological agents in trials (Rahman K (November 2007). “Effects of garlic on platelet biochemistry and physiology”. Mol Nutr Food Res 51 (11): 1335-44. doi:10.1002/mnfr.200700058.PMID 17966136. Mader F H (October 1990). “Treatment of hyperlipidaemia with garlic-powder tablets. Evidence from the German Association of General Practitioners' multicentric placebo-controlled double-blind study”. Arzneimittelforschung 40 (10): 1111-6. PMID 2291748).

The cloves of Garlic have been used for the indications of problematic fungal infections like Thrush or Candidiasis (Lemar K M, Passa O, Aon M A, et al. (October 2005). “Allyl alcohol and garlic (Allium sativum) extract produce oxidative stress in Candida albicans”. Microbiology (Reading, Engl.) 151 (Pt 10): 3257-65.doi:10.1099/mic.0.28095-0. PMC 2711876.PMID 16207909.).

Also, like other herbs mentioned earlier in this text, Garlic has been used as a preservative for meats because its antimicrobial effects are sustained at high temperatures (Ranjan, Shivendu et al. (2012). “Comparative study of antibacterial activity of garlic and cinnamon at different temperature and its application on preservation of fish”. Adv. Appl. Sci. Res 3 (1): 495-501.).

The compounds found in Garlic that provide beneficial effects for culinary use and possible scientific applications are numerous. The high sulfur content of many species of Garlic gives its notable odor and side effects. The main compounds that have been noted thus far are Allicin, Alliin, Thiosulfinates, Dially-tri sulfide, Dially-di sulfide, Diallyl sulfide, Ajoene, S-Allycysteine and other active sulfur based compounds.

The most popularly researched and discussed compound in Garlic is Allicin. Allicin is found when raw garlic cloves have been chopped, chewed and/or crushed. The process in which this occurs is a very important process. When crushed or pressed, enzyme catalysts like Allinase are released which then initiates sulfenic acids to react with one another. This process can take a short time of only 10-60 seconds. Allicin will continue to break down to other sulfur compounds that are listed above. These collective compounds reacting together are what give Garlic its potential properties in contemporary research.

In the purpose of this text, Garlic is referenced for its potential antimicrobial effects as well as its potential anti-inflammatory effects. Specifically, it is referenced in this text for its action against strains of Mycobacteria and the related initiation of pro-inflammatory cytokines that are involved. The specific pathogen as it relates to this application is Mycobacterium subspecies paratuberculosis (MAP). Garlic has been researched and shown to lower or in some manner inhibit the pro-inflammatory enzymes cyclooxygenase (COX-2) and lipoxygenase (5-LOX) (Ali M, Thomson M, Afzal M. Garlic and onions: their effect on eicosanoid metabolism and its clinical relevance. Prostaglandins Leukot Essent Fatty Acids. 2000; 62(2):55-73. (PubMed).

Garlic has been tested against a variety of mycobacteria and has shown in vitro inhibition depending on dosage and culture time (J. Antimicrob. Chemother. (1993)32 (4): 623-626.doi: 10.1093/jac/32.4.623). In a trial involving patients with MAC pulmonary infections, Garlic was tested against 18 strains of Mycobacterium avium and showed inhibitory effects in vitro (J. Antimicrob. Chemother. (1993)32 (4): 623-626.doi: 10.1093/jac/32.4.623). This is important as persons with autoimmune conditions are hypothesized to have latent mycobacterial infections as suspect to their related conditions. The main bacterium that is suspected is MAP, a close relative of M. avium which shows many similarities in their respective behaviors. MAP is strongly suspected in cases of collective conditions known as inflammatory bowel disease. With multiple studies showing Garlic extract to display inhibitory effects against mycobacteria, it is important to consider using an agent that has a variety of positive benefits for patients that shows strong antimicrobial, antioxidant (inducing glutathione synthesis), and possible apoptosis against tumor cells (Chen C, Pung D, Leong V, et al. Induction of detoxifying enzymes by garlic organosulfur compounds through transcription factor Nrf2: effect of chemical structure and stress signals. Free Radic biol Med. 2004; 37(10):1578-1590. (PubMed) (Herman-Antosiewicz A, Singh S V. Signal transduction pathways leading to cell cycle arrest and apoptosis induction in cancer cells by Allium vegetable-derived organosulfur compounds: a review. Mutat Res. 2004; 555(1-2):121-131. (PubMed).

In addition to a wide array of other infectious agents, garlic extract has been and is still used for its antimicrobial effects. This is largely due to the content and amount of allicin in each species of garlic. The derivatives of allicin and other high sulfur based compounds found in garlic are what may be of benefit in treating infectious conditions that range from a wide array of pathogens. Garlic extract has a multitude of actions against pathogens most likely due to the many different compounds found in dry extract, raw cloves, and aged preparations.

Garlic compounds would be utilized in the proposed product for their possible benefits in the treatment of autoimmune conditions as described in the text above. Patients with autoimmune conditions usually have decreased levels of immune function, especially when the adjunct compounds used in allopathic medicine aim to primarily lower immune function based on lowering inflammatory markers. While in acute settings, the utilization of such synthetics may be warranted to shift the biological terrain. In long term therapy, agents that lower immune function with the goal of decreasing inflammation, tend to leave patients prone to secondary infections and low levels of antioxidant compounds like glutathione and superoxide dismutase (SOD). The low levels of antioxidant compounds can, in turn, play a role in the possible development of other conditions. Garlic and its derivatives have the potential to increase antioxidant levels mentioned herein (glutathione and SOD), display a wide range antimicrobial action against suspected agents that may play a role in initiating autoimmune conditions, and display potential for anti-inflammatory action—all without leaving patients prone to other infections. The compounds suggested show action of working with biological functions that can lower patients' inflammatory markers, have secondary increased output of much needed antioxidant levels, while possibly improving quality of life.

Oregon Grape (Mahonia aquifolium) 200 mg yielding active compounds of Berberine, Jatorrhizine and Magnoflorine.

Mahonia aquifolium or Oregon Grape is a plant of flowering nature that is classified in the family of Berberidaceae. The plants found in this family have been used for many years for their naturally high content of the compounds berberine, jatorrhizine, and magnoflorine. The most researched of these compounds has been to date, berberine. Berberine is an alkaloid found in high quantities in other plants related to Oregon Grape like Berberis vulgaris (Barberry) and Hydrastis Canadensis (Goldenseal). Berberine has been researched for many decades for treatment of fungal infection, protozoan leishmaniasis, anti-inflammatory effects via suppression of cytokines, effects on fatty acid metabolism via adiponectin, diabetes, cancer, liver function, cardiovascular conditions, and possible mental health benefits.

Oregon grape is an evergreen shrub that as its name states, is commonly found in the northwestern part of the United States and is native to Alaska, Oregon, Washington, California, Colorado and parts of western Canadian territories. It has been cultivated in many other areas of the nation as it is used as a secondary agent taking the place of using the endangered Goldenseal species. It is a close relative to that of common Barberry as well as the over-harvested Goldenseal. It has leaves that have edges resembling that of common Holly species. It produces flowers along with berries that are sometimes used in culinary and herbal preparations. Usually the use of berries in raw form produces a very bitter tasting product so it is not the best utilization of the plant. Oregon Grape can grow from 3-6 feet tall, and sometimes up to 7 feet tall.

For the stated purpose of this invention, the compounds, mainly berberine are intended to be utilized as it has displayed strong antimicrobial effects and selective action against suspected co-infections that may be related to autoimmune disorders. Suspected pathogens have been purported with autoimmune disorders as initiators of chronic inflammatory responses (Chiodini, R., Chamberlin, W., Sarosiek, J., & McCallum, R. (2012). Crohn's disease and the mycobacterioses: A quarter century later. Causation or simple association? Divisions of Infectious Diseases, 38(1), 52-93.). The main agent that has been recently researched is Mycobacterium avium subspecies paratuberculosis (MAP). The question has been investigated that other pathogens like that of Staphylococcus family, E. coli family pathogens, and other co-infectors like that of Candida albicans may be related to MAP infections. There is an open area of research that needs to focus more on the topic of co-infections and MAP provides a window to that area.

The other relation to this invention that Oregon Grape and the compound berberine provide are that of decreasing bacterial resistance to selected antibiotic compounds. Oregon Grape and its famous active compound, berberine, have been found to produce a multi-drug resistance pump inhibitor or MDR compound. This has been classified as 5-methoxyhydnocarpin or 5-MHC. Bacterium can produce defenses against agents over time that are classified as ubiquitous multidrug resistance pumps. Areas on bacterial membrane recognize and remove substances foreign to the pathogen via these MDR pumps. The 5-MHC compound found in plants containing berberine has a substrate that inhibits the cations that start the removal of antimicrobial agents of natural and synthetic origin (Stermitz F R, Lorenz P, Tawara J N, Zenewicz L A, Lewis K (February 2000). “Synergy in a medicinal plant: antimicrobial action of berberine potentiated by 5′-methoxyhydnocarpin, a multidrug pump inhibitor”. Proc. Natl. Acad. Sci. U.S.A. 97 (4): 1433-7.doi:10.1073/pnas.030540597. PMC 26451.PMID 10677479). This is extremely important for the treatment of pathogens that are in the family of Mycobacterial origin. Mycobacteria have been known throughout their long history and relationship with science to become resistant to contemporary agents that are used against them. This is widely obvious with the issues of MDR tuberculosis and XDR TB. With the suggestion that MAP may play an important role in autoimmune disease, it is again concerning that treatment of the pathogen could result in resistance to selected antimicrobial agents if not used correctly.

Oregon Grape also displays beneficial treatment of skin disorders like psoriasis, eczema, and contact dermatitis (Rackova L, Oblozinsky M, Kostalova D, Kettmann V, Bezakova L (2007). “Free radical scavenging activity and lipoxygenase inhibition of Mahonia aquifolium extract and isoquinoline alkaloids”. J Inflamm (Lond) 4: 15. doi:10.1186/1476-9255-4-15. PMC 1994948.PMID 17634120.).

Another important benefit of Oregon Grape and use in relation to this text, is its ability to assist with digestive discomfort. The issues that have been associated with autoimmune conditions (specifically related to inflammatory bowel disease) are chronic diarrhea, dyspepsia, distention that leads to intermittent discomfort or pain, and spasmodic episodes. The symptoms discussed can lead to lower quality of life and loss of appetite and absorption of certain nutrients. Oregon Grape has been used for years as a “bitter” which is traditionally used to help alleviate digestive concerns. This is an important factor to consider in the treatment of autoimmune conditions, most importantly IBD, which should utilize the possible benefits that Oregon Grape and berberine have displayed in previous scientific studies. Several studies show that the alkaloids active in Oregon Grape may be useful in treating these concerns (Tewari J P, Srivastava M C, Bajpai J L. Pharmacologic studies of Achillea millefolium Linn. Indian J Med Sci 1994; 28(8):331-6.) (Rabbani G H, Butler T, Knight J, et al. Randomized controlled trial of berberine sulfate therapy for diarrhea due to enterotoxigenic Escherichia coli and Vibrio cholerae. J Infect Dis 1987; 155:979-84.) (Eaker E Y, Sninsky C A. Effect of berberine on myoelectric activity and transit of the small intestine in rats. Gastroenterol 1989; 96:1506-13.).

To summarize the beneficial properties of Oregon Grape, the active compounds that are found in the plant have displayed effectiveness in the issues that arise with autoimmune conditions, especially inflammatory bowel disease. The active constituents found in Oregon Grape may be useful in the problematic area of bacterial resistance. This may be directly related to autoimmune conditions as the suspected role of pathogens, particularly Mycobacterium avium subspecies paratuberculosis (MAP), are currently being researched. It is suggested that this strain(s) of pathogen can be difficult in treating as stated, because resistance may be of concern. Oregon Grape and berberine may have an important role in addressing the issue of resistance of synthetic and natural antimicrobial agents. Oregon Grape has been used for many other infectious agents that may be co-infections of strong pathogen like MAP. Oregon Grape may also be beneficial in symptomatic treatment that is prevalent in conditions such as inflammatory bowel disease. These examples are important as contemporary modalities have repeatedly yielded low benefit to persons with autoimmune conditions.

Boswellia serrata 300 mg Yielding Acetyl-11-Keto-Beta-Boswellic Acid.

Boswellia serrata (Salai guggal) is a tree of the Sapindales family that also contains well known trees such as chestnut, maple, cashew, citrus, and mahogany. Boswellia is widely known as Frankincense in many countries throughout history. As with many herbs, Boswellia's use has dated back to Biblical times. Boswellia was initially used for its fragrance and aroma but as its use continued through time, it became widely pronounced for its anti-inflammatory properties and analgesic action.

Boswellia is usually found as a small to medium sized tree or shrub. It is native to the regions of Asia and Africa. The highest percentage of the plant is found in India and central to northern Africa. The species of Boswellia plants are generally grown best in areas that are not prone to seasonal changes and are usually of a more humid than dry climate. As research and science recently has shown its benefits, the plant has been cultivated in many other areas throughout the world. Modern technological advances allow for it to be cultivated even out of its native climate.

The main actions and mechanisms that are explained for the purpose of this text and related product are Boswellia's anti-inflammatory properties. The related conditions, that this text and compounds aim to address, are those labeled as autoimmune. The main conditions that are most applicable for Boswellia's beneficial properties are Rheumatoid arthritis, Crohn's disease, and ulcerative colitis. These conditions usually are treated in the same manner in allopathic medicine with the same class of prescribed drugs. The use of immune modulating agents like azathioprine, infliximab, and corticosteroids are standard in treatment protocols for these conditions. The long-term use of these agents can bring secondary concerns or conditions for patients who have been prescribed to take them for long durations. Long term use may lead to infections from suppressing the immune system and possible related osteopathic issues from use of corticosteroids, among many others. The options for patients are currently limited and this projected product is aimed to address these concerns.

The compounds found in the resin of Boswellia serrata species plants have been demonstrated in several scientific studies for patients with inflammatory bowel disease (Gupta I, Parihar A, Malhotra P, et al. Effects of Boswellia serrata gum resin in patients with ulcerative colitis. Eur J Med Res. 1997; 2:37-43) (Safayhi H, Sailer E R, Ammon H P T. 5-lipoxygenase inhibition by acetyl-11-keto-beta-boswellic acid (AKBA) by a novel mechanism. Phytomedicine. 1996; 3:71-72.) (Safayhi H, Boden S E, Schweizer S, et al. Concentration-dependent potentiating and inhibitory effects of Boswellia extracts on 5-lipoxygenase product formation in stimulated PMNL. Planta Med. 2000; 66:110-113.) (Kimmatkar N, Thawani V, Hingorani L, et al. Efficacy and tolerability of Boswellia serrata extract in treatment of osteoarthritis of knee—a randomized double blind, placebo controlled trial. Phytomedicine. 2003; 10:3-7.).

The pro-inflammatory compound that is found to be elevated in persons with autoimmune conditions (5-lipoxygenase) was found to be influenced by a compound found in Boswellia named acetyl-11-keto-beta-boswellic acid. 5-lipoxygenase is a strong enzyme that starts the formation of leukotrienes. In studies referenced, when the active compounds found in Boswellia were researched in comparison to sulfasalazine (a common anti-inflammatory agent used as a control), the group who received Boswellia gum resins showed remission rates higher (82%) than the control group (75%) (Safayhi H, Boden S E, Schweizer S, et al. Concentration-dependent potentiating and inhibitory effects of Boswellia extracts on 5-lipoxygenase product formation in stimulated PMNL. Planta Med. 2000; 66:110-113).

The acids contained in the Boswellia species have also been shown to influence interleukin-1 beta in a positive manner. IL-1(3 is one of the key inflammatory cytokines in autoimmune disease and is highly involved in inflammatory bowel disease. The suggestion to utilize compounds found in Boswellia for said conditions may give patients an option for treatment methods that yield positive results. Boswellia and its active acids may also show potential in the treatment of tumor formation as well. This is important to note for autoimmune patients as it has been suggested that chronic inflammatory action can lead to development of neoplastic formations. The action that Boswellia displays in this area may be due to blunting of elastase in leukocytes and induction of apoptosis (Safayhi H, Rall B, Sailer E R, and Ammon H P. Inhibition by boswellic acids of human leukocyte elastase. J Pharmacol Exp Ther 281: 460-463, 1997).

In addition to acetyl-11-keto-beta-boswellic acid, there are many other active compounds that have been found in Boswellia serrata species. Among the most prominent are compounds consisting of oleoresins, sesquiterpenoid oils, and ursane-pentacyclic triterpenes. These have been shown to be the active compounds in Boswellia that are researched and isolated for their effects (Safayhi H, Mack T, Sabieraj J, Anazodo M I, Subramanian L R, and Ammon H P. Boswellic acids: novel, specific, nonredox inhibitors of 5-lipoxygenase. J Pharmacol Exp Ther 261: 1143-1146, 1992).

Boswellia is a potential alternative and has been looked to in the “Complementary and Alternative” medicine arena for many years now to help address many facets of inflammatory conditions. It has been used in Ayurveda for the same conditions many decades before the current scientific studies have displayed the compounds active in these processes. The suggested use along with the other compounds and agents featured in this text is to address the issue of chronic inflammation that may be related to mycobacterial infection, specifically Mycobacterium avium subspecies paratuberculosis. It is hypothesized that inflammation is a secondary action related to a suspected infectious agent such as MAP. The possible mechanisms and action that Boswellia displays in research, recent and historically, would suggest its use as a potential agent for autoimmune conditions and their related events. Furthermore, Boswellia and its compounds may show potential action against tumor formation along with the natural process of displaying its analgesic properties as a GRAS compound.

### EXAMPLES

In the following examples the invention is elucidated in the subsequent list (without being limited to those contained herein).

The invention will be comprised of 300 milligrams of Cinnamon oil and chemistry and compounds found therein, 150 milligrams of Origanum vulgare oil chemistry and compounds found therein, 200 milligrams of Centella asiatica chemistry and compounds found therein, 4000 international units of Vitamin A and related carotenoids and compounds found therein, 1500 international units of Vitamin D3 as cholecalciferol and its compounds found therein, 400 milligrams of Allium sativum chemistry and compounds found therein, 200 milligrams of Mahonia aquifolium chemistry and compounds found therein, 300 milligrams of Boswellia serrata and its chemistry and compounds found therein.

### Example 1—Soft-Gelatin Encapsulation

The synergistic effect of each ingredient of the composition are found as 300 mg Cinnamon oil, 150 mg of Oregano oil, 200 mg of Centella asiatica, 4000 IU of Vitamin A, 1500 IU of Vitamin D3, 400 mg of Allium sativum, 200 mg of Mahonia aquifolium and 300 mg of Boswellia serrata. The intention of the synergy between all compounds in the invention are directed to enhance immune function, lower inflammation and address suspected infection of Mycobacterium avium sub-species paratuberculosis. Whereas the term soft-gelatin capsules describe encapsulation products and procedures meant for consumption and delivery of said compounds. The capsules are to be ingested as one dose or divided doses depending on manufacturing specifications.

### Example 2—Enteric Coated Gelatin Capsule

The composition as described in Example 1 remains the same whereas substitution will be made to utilize Enteric coated capsules comprised of vegetable cellulose or gelatin. The intent to utilize Enteric coated capsules are meant as to ensure proper delivery of ingredients to the digestive tract without being compromised by pyloric acids found in the upper digestive areas. Enteric coated capsules are described as encapsulation products and procedures meant for consumption and delivery of said compounds. The capsules are to be ingested as one dose or divided doses depending on manufacturing specifications.

### Example 3—Vegetable Cellulose Capsules

The composition as described in Example 1 remains the same whereas substitution will be made to utilize vegetable cellulose capsules. The intention to utilize vegetable cellulose capsules are to offer a delivery system that allows persons who do not consume animal products due to allergy, safety or religion practices to utilize said invention without the concerns mentioned. Vegetable cellulose capsules are described as encapsulation products meant for consumption and delivery of said compounds. The capsules are to be taken in one dose or divided doses depending on manufacturing specifications.

### Example 4—Liquid Tincture of all Extracts

The composition as described in Example 1 remains the same whereas substitution will be made to use liquid extracts of all ingredients of said invention. The term liquid extract is described as taking the active compounds from all listed ingredients of said invention via methods of hot water extraction or alcohol extraction and placing them into a stabilized tincture to be used best by placing finalized solution into a liquid carrier, usually purified water, to be ready for consumption. The suggested dose will range from one dose or divided doses depending on manufacturing specifications.

### Example 5—Pre-Mixed Solution

The composition as described in Example 1 remains the same whereas substitution will be made to use a pre-mixed powder that can be dissolved in a liquid carrier, preferably purified water. The suggested dose will range from one dose or divided doses depending on manufacturing specifications.

### Example 6—Tablets

The composition as described in Example 1 remains the same whereas substitution will be made to use a tablet formed delivery system. The compounds listed in the invention can be placed into powder form then formed into tablets to offer convenience for persons needing to ingest tablets rather than a pre-mixed solution as described in Example 5. The dose will range from one dose or divided doses depending on manufacturing specifications.

### Example 7—Interchangeable Delivery Methods

The composition as described in Example 1 remains the same whereas any method described in Examples 1-6 may be substituted or interchanged with each other depending on manufacturing specifications.

What is claimed is:

1. We claim that the synergistic action of the compounds contained in the invention—the naturally occurring compounds from the botanicals Cinnamon oil at 300 mg of hydro or ethanolic extract yielding the active compounds found in Cinnamomum species—cinnamaldehyde, trans-cinnamaldehyde, ethyl cinnamate, eugenol and linalool: the naturally occurring compounds from Oregano oil at 150 mg of hydro or ethanolic extract yielding the active compounds found in O. vulgare—carvacrol, thymol, caryophyllene, spathulenol and beta-fenchyl; the naturally occurring compounds from Garlic at 400 mg of hydro or ethanolic extract yielding the active compounds found in Allium sativum-allicin, alliin, s-allyl -cysteine, ajoene, diallyl disulfide and various thiosulfinates. the naturally occurring compounds found in Gotu Kola at 200 mg of hydro or ethanolic extract yielding the active compounds found in C. asiatica—asiaticoside, asiaticoside-B, madecassoside, Asiatic acids, madecassic acid: the naturally occurring compounds found in Oregon Grape at 200 mg of hydro or ethanolic extract yielding the active compounds found in Mahonia aquifolium-berberine, jatrorrhizine and magnoflorine; the naturally occurring compounds found in Boswellia serrata at 300 mg of hydro or ethanolic extract yielding the active compounds found in B. serrata-11-keto-beta-boswellic acid, oleoresins, sesquiterpenes and triterpenes; the fat soluble nutrient Vitamin A at 4,000 IU in liquid form or powder form; the fat soluble nutrient Vitamin D3 at 1500 IU in liquid or powder form; these compounds and chemicals collectively will address the hypothesized infection on Mycobacterium paratuberculosis, lower inflammatory markers and support immune function of persons with autoimmune conditions of inflammatory bowel disease (IBD), Type 1 diabetes (T1DB), Multiple Sclerosis and Psoriasis among others.

2. We claim that the invention will be utilized best by the delivery methods of soft gelatin capsules, vegetable cellulose capsules, liquid capsules, liquid tincture, powder, tablet or any combination thereof.

3. We claim that the invention will offer a treatment option that does not create the same side events and effects for persons with autoimmune conditions especially those of IBD, MS, T1DB and Psoriasis as conventional treatment such as corticosteroids, TNF-α blocking agents and purine inhibiting agents may create additional negative health outcomes needing further treatment; the invention will address lack of access to treatment due to rising costs of the conventional treatments while simultaneously addressing the issues of providing methods of improved outcomes in autoimmune conditions in the areas of potential infection, inflammation and immune function.

4. The invention as described in claim 1 will be aimed at the natural products industry and alternative health marketplace to create accessibility to the invention and offer a solid foundation of a treatment plan for persons with autoimmune conditions mentioned herein; the potential outreach of the invention for public use in addition to retailers shall be aimed at alternative health practitioners who already have a foundation of understanding in the alternative treatment modalities.

5. The invention as described in claim 1 will have potential use in the veterinary area of medicine as the compounds listed in the invention address the issue of Mycobacterium paratuberculosis (MAP) and its role in Johne's disease in ruminants.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)
